(19) United States
(12) Patent Application Publication
(10) Pub. No.: US 2014/0193882 A1
(43) Pub. Date: Jul. 10, 2014

(54) NOVEL STRAINS OF BACTERIOPHAGES FOR THE TREATMENT OF BACTERIAL INFECTIONS, PARTICULARLY BY STRAINS OF DRUG-RESISTANT BACTERIA OF THE GENUS STENOTROPHOMONAS

(71) Applicants: Beata Weber-Dabrowska, Wroclaw (PL); Andrzej Gorski, Wroclaw (PL); Marzena Lusiak-Szelachowska, Wroclaw (PL); Maciej Zaczek, Wroclaw (PL)

(72) Inventors: Beata Weber-Dabrowska, Wroclaw (PL); Andrzej Gorski, Wroclaw (PL); Marzena Lusiak-Szelachowska, Wroclaw (PL); Maciej Zaczek, Wroclaw (PL)

(73) Assignee: INSTYTUT IMMUNOLOGII I TERAPII DOSWIADCZALNEJ PAN, Wroclaw (PL)

(21) Appl. No.: 13/805,408

(22) PCT Filed: Jun. 20, 2011
(86) PCT No.: PCT/PL11/50025

§ 371 Date: Oct. 11, 2013

(30) Foreign Application Priority Data
Jun. 20, 2010 (PL) P.391552

(51) Int. Cl. C12N 7/00

(52) U.S. Cl. CPC C12N 7/00
USPC 435/235.1

(57) ABSTRACT

Bacteriophage strains and their use in the production of preparations for use in the treatment of bacterial infections, particularly by strains of drug-resistant bacteria of the genus Stenotrophomonas, particularly those belonging to the species Stenotrophomonas maltophilia

### BACKGROUND OF THE PRESENT INVENTION

The present invention relates to novel strains of bacteriophages and their use in the treatment of bacterial infections, particularly by strains of drug-resistant bacteria of the genus Stenotrophomonas.

### STATE OF THE ART

Bacteriophages (phages) constitute a varied group of viruses, whose life cycle is connected solely with bacterial cells. Bacteriophages are characterised by a lytic or lysogenic life cycle. Primarily lytic bacteriophages are useful as antibacterial agents, which multiply following the inoculation of cells sensitive to them, causing their complete lysis, and the released novel phage molecules attack and destroy subsequent bacterial cells. This process can occur both in vitro and in vivo.

One of the significant characteristics of bacteriophages is the commonly known high specificity of their lytic activity. This characteristic is used, amongst others, in typing various bacterial species (for examples, see patent descriptions GB 2285684, U.S. Pat. No. 5,824,468, SU 543260 and international patent applications WO 0100786, WO 0109370). Other known uses of bacteriophages encompass: the use of bacteriophages as tools in molecular biology useful, for example, in the expression and selection of desirable proteins (i.e. patent description U.S Pat. No. 6,027,930), the use of phages in sterilizing and cleaning agents (i.e. patent description EP 0414304, EP 0290295, GB 2253859 and international patent applications WO 9808944, WO 9003122). Modified phages have been used in the production of vaccines (i.e. WO 9505454). Some bacteriophage proteins are also used (i.e. EP 0510907, U.S. Pat. No. 5,470,573, WO 9607329). Methods of isolating bacteriophages and of producing phage preparations are well known and are continually being improved (i.e. GB 829266, CS 192212, RU 2109055).

On a broad scale, phage therapy has been in use since WWII at the Microbiology and Virology Institute in Tbilisi (Georgia). Pools of various phage preparations are used there in the treatment of bacterial infections and in prophylaxis. Available data show the large effectiveness of phage therapy. Similar research has been conducted for years in Poland, at the Bacteriophage Laboratory of the Institute of Immunology and Experimental Therapy of the Polish Academy of Sciences in Wroc/law, where phage therapy of infections by bacteria that are drug-resistant and unresponsive to antibiotics has been conducted for over 25 years (see: Stefan Ślopek et al., Archivum Immunologiae et Therapiae Experimentalis, 1981, 31, 293; Stefan Ślopek et al., Archivum Immunologiae et Therapiae Experimentalis, 1983, 31, 267; Stefan Ślopek et al., Archivum Immunologiae et Therapiae Experimentalis, 1984, 32, 317; Stefan Ślopek et al., Archivum Immunologiae et Therapiae Experimentalis, 1985, 33, 219; Stefan Ślopek et al., Archivum Immunologiae et Therapiae Experimentalis, 1985, 33, 241; Stefan Ślopek et al., Archivum Immunologiae et Therapiae Experimentalis, 1987, 35, 569; Beata Weber-D [symbol] browska et al., Archivum Immunologiae et Therapiae Experimentalis, 2000, 48, 31-37; Beata Weber-D [symbol] browska et al., Archivum Immunologiae et Therapiae Experimentalis, 2000, 48, 547-551).

Patent application P-370662 relates to polyvalent bacteriophage strains, methods of producing them and their use in the treatment of bacterial infections, particularly by strains of drug-resistant bacteria of the genera Pseudomonas and Staphylococcus.

Particular problems are caused by blood infections, pneumonias, and urinary tract infections caused by drug-resistant bacteria, most often of the genus Stenotrophomonas. Treatment of such infections with the available antibiotics, particularly in recent years, causes significant therapeutic problems worldwide. The antibiotic therapy of these infections is becoming increasingly ineffective, because a great majority of the strains of these bacteria demonstrates a natural resistance to all antibiotics, including the last ditch antibiotic, vancomycin. There is thus a deep need for the introduction into practical medicine of an alternative method of treating persistent and very dangerous bacterial infections.

### GOAL OF THE PRESENT INVENTION

The last several years have seen a massive dispersal of bacterial strains resistant to all antibiotics, including the last-ditch antibiotic vancomycin. As a result, the treatment of bacterial infections induced by drug-resistant forms with antibiotics is ineffective.

This state of affairs causes drastic therapeutic problems. There is thus an urgent need for the introduction of alternative methods of treating persistent bacterial infections into medical praxis.

In light of the above, the goal of the present invention is to obtain polyvalent phage preparations, which could be effectively used in the treatment of bacterial infections caused by drug-resistant clinical strains of bacteria of the genus Stenotrophomonas, in particular those belonging to the species Stenotrophomonas maltophilia.

Unexpectedly, the above defined goal has been attained by the present invention.

The subject of the present invention is bacteriophage strain specific against bacteria belonging to the genus Stenotrophomonas deposited in the Polish Microbiological Collection under the accession number PCM F/00046.

The next subject of the present invention is the use of a bacteriophage strain deposited in the Polish Microbiological Collection under the accession number PCM F/00046 in the production of antibacterial preparations for use in combating bacteria belonging to the genus Stenotrophomonas. Preferably, the counteracted bacteria belong to the species Stenotrophomonas maltophilia.

Another subject of the present invention is a bacteriophage strain specific against bacteria belonging to the genus Stenotrophomonas deposited in the Polish Microbiological Collection under the accession number PCM F/00047

The next subject of the present invention is the use of a bacteriophage strain deposited in the Polish Microbiological Collection under the accession number PCM F000/47 in the production of antibacterial preparations for use in combating bacteria belonging to the genus Stenotrophomonas. Preferably, the counteracted bacteria belong to the species Stenotrophomonas maltophilia.

Another subject of the present invention is a bacteriophage strain specific against bacteria belonging to the genus Stenotrophomonas deposited in the Polish Microbiological Collection under the accession number PCM F/00048

The next subject of the present invention is the use of a bacteriophage strain deposited in the Polish Microbiological Collection under the accession number PCM F/00048 in the production of antibacterial preparations for use in combating bacteria belonging to the genus Stenotrophomonas. Preferably, the counteracted bacteria belong to the species Stenotrophomonas maltophilia.

Another subject of the present invention is a bacteriophage strain specific against bacteria belonging to the genus Stenotrophomonas deposited in the Polish Microbiological Collection under the accession number PCM F/00049

The next subject of the present invention is the use of a bacteriophage strain deposited in the Polish Microbiological Collection under the accession number PCM F/00049 in the production of antibacterial preparations for use in combating bacteria belonging to the genus Stenotrophomonas. Preferably, the counteracted bacteria belong to the species Stenotrophomonas maltophilia.

Another subject of the present invention is a bacteriophage strain specific against bacteria belonging to the genus Stenotrophomonas deposited in the Polish Microbiological Collection under the accession number PCM F/00050.

The next subject of the present invention is the use of a bacteriophage strain deposited in the Polish Microbiological Collection under the accession number PCM F/00050 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00051.

The next subject of the present invention is the use of a bacteriophage strain deposited in the Polish Microbiological Collection under the accession number PCM F/00051 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00052

The next subject of the present invention is the use of a bacteriophage strain deposited in the Polish Microbiological Collection under the accession number PCM F/00052 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00053

The next subject of the present invention is the use of a bacteriophage strain deposited in the Polish Microbiological Collection under the accession number PCM F/00053 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCMF/00054.

The next subject of the present invention is the use of a bacteriophage strain deposited in the Polish Microbiological Collection under the accession number PCM F/00054 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00055

The next subject of the present invention is the use of a bacteriophage strain deposited in the Polish Microbiological Collection under the accession number PCM F/00055 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00056

The next subject of the present invention is the use of a bacteriophage strain deposited in the Polish Microbiological Collection under the accession number PCM F/00056 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00057

The next subject of the present invention is the use of a bacteriophage strain deposited in the Polish Microbiological Collection under the accession number PCM F/00057 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00058.

The next subject of the present invention is the use of a bacteriophage strain deposited in the Polish Microbiological Collection under the accession number PCM F/00058 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00059.

The next subject of the present invention is the use of a bacteriophage strain deposited in the Polish Microbiological Collection under the accession number PCM F/00059 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00060.

The next subject of the present invention is the use of a bacteriophage strain deposited in the Polish Microbiological Collection under the accession number PCM F/00060 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00061.

The next subject of the present invention is the use of a bacteriophage strain deposited in the Polish Microbiological Collection under the accession number PCM F/00061 in the production of antibacterial preparations for use in combating bacteria belonging to the genus Stenotrophomonas. Preferably, the counteracted bacteria belong to the species Stenotrophomonas maltophilia.

The subject of the present invention is a bacteriophage strain specific against bacteria belonging to the genus Stenotrophomonas deposited in the Polish Microbiological Collection under the accession number PCM F/00062.

The next subject of the present invention is the use of a bacteriophage strain deposited in the Polish Microbiological Collection under the accession number PCM F/00062 in the production of antibacterial preparations for use in combating bacteria belonging to the genus Stenotrophomonas. Preferably, the counteracted bacteria belong to the species Stenotrophomonas maltophilia.

### EXAMPLE 1

### Stenotrophomonas maltophilia Phage 1B/145

Virulent bacteriophage isolated from raw sewage from the River Port in Wroc/law on S. maltophilia strain 145.

Phage isolation was carried out after 1 h of incubation at a temperature of 37° C. from the examined sewage sample in a liquid nutritive medium (at a 1:1 ratio) with the addition of 0.1 ml of a fresh culture of the S. maltophilia strain 145. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 145 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain was deposited on Jun. 16, 2010 in the Polish Microbiological Collection, which possesses the international deposit authority status for the purposes of patent proceedings (IDA). The patent deposit was given the accession number PCM F/00046.

### EXAMPLE 2

### Stenotrophomonas maltophilia Phage 2B/SK1

Virulent bacteriophage isolated from irrigated fields in Wroc/law on S. maltophilia strain SKI1.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain SK1. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain SK1 and incubated for 18 h at a temperature of 37° C. The resulting phage lysate was centrifuged for 30 min. at 4.5 KRPM and passed through antibacterial filters, and controlled for the presence of the phage.

The Stenotrophomonas maltophilia phage strain 2B/SK1 was deposited on Jun. 16, 2010 in the Polish Microbiological Collection, which possesses the international deposit authority status for the purposes of patent proceedings (IDA). The patent deposit was given the accession number PCM F/00047

### EXAMPLE 3

### Stenotrophomonas maltophilia Phage 3/SK1

Virulent bacteriophage isolated from a sample of water from the Red Sea on S. maltophilia strain SK1.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain SK1. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain SK1 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 3/SK1 was deposited on Jun. 25, 2007 in the Polish Microbiological Collection, which possesses the international deposit authority status for the purposes of patent proceedings (IDA). The patent deposit was given the accession number PCM F/00048

### EXAMPLE 4

### Stenotrophomonas maltophilia Phage 4/SK1

Virulent bacteriophage isolated from the raw sewage of a sewage treatment plant in Strachocin and in Wroc/law on S. maltophilia strain SK1.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain SK1. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain SK1 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain was deposited on Jun. 16, 2010 in the Polish Microbiological Collection, which possesses the international deposit authority status for the purposes of patent proceedings (IDA). The patent deposit was given the accession number PCM F/00049

### EXAMPLE 5

### Stenotrophomonas maltophilia Phage 5/SK1

Virulent bacteriophage isolated from raw sewage of the treatment plant Strachocin II in Wroc/law on S. maltophilia strain SK1.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain SK1. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain SK1 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 5/SK1 was deposited on Jun. 16, 2010 in the Polish Microbiological Collection, which possesses the international deposit authority status for the purposes of patent proceedings (IDA). The patent deposit was given the accession number PCM F/00050

### EXAMPLE 6

### Stenotrophomonas maltophilia Phage 6/SK1

Virulent bacteriophage isolated from sewage from the River Port in Wroc/law on S. maltophilia strain SK1.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain SK1. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain SK1 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 6/SK1 was deposited on Jun. 16, 2010 in the Polish Microbiological Collection, which possesses the international deposit authority status for the purposes of patent proceedings (IDA). The patent deposit was given the accession number PCM F/00051.

### EXAMPLE 7

### Stenotrophomonas maltophilia Phage 7/316

Virulent bacteriophage isolated from raw sewage from the River Port in Wroc/law on S. maltophilia strain 316.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 316. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 316 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 7/316 was deposited on Jun. 16, 2010 in the Polish Microbiological Collection, which possesses the international deposit authority status for the purposes of patent proceedings (IDA). The patent deposit was given the accession number PCM F/00052.

### EXAMPLE 8

### Phage Stenotrophomonas maltophilia 8/321

Virulent bacteriophage isolated from raw sewage from the River Port in Wroc/law on S. maltophilia strain 321

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 321. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 321 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 8/321 was deposited on Jun. 16, 2010 in the Polish Microbiological Collection, which possesses the international deposit authority status for the purposes of patent proceedings (IDA). The patent deposit was given the accession number PCM F/00053

### EXAMPLE 9

### Phage Stenotrophomonas maltophilia 9/322

Virulent bacteriophage isolated from irrigated fields in Wroc/law on S. maltophilia strain 322.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 322. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 322 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 9/322 was deposited on Jun. 16, 2010 in the Polish Microbiological Collection, which possesses the international deposit authority status for the purposes of patent proceedings (IDA). The patent deposit was given the accession number PCM F/00054

### EXAMPLE 10

### Phage Stenotrophomonas maltophilia 10/535

Virulent bacteriophage isolated from raw sewage from a sewage treatment plant in Opole on Stenotrophomonas maltophilia strain 535.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 535. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 535 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 10/535 was deposited on Jun. 16, 2010 in the Polish Microbiological Collection, which possesses the international deposit authority status for the purposes of patent proceedings (IDA). The patent deposit was given the accession number PCM F/00055

### EXAMPLE 11

### Stenotrophomonas maltophilia Phage 11/8c

Virulent bacteriophage isolated from raw sewage from a sewage treatment plant in Wroc/law on S. maltophilia strain 8c.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 8c. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 8c and incubated for 18 h at 37° C. The resulting phage lysate was centrifuged for 30 min. at 4.5 KRPM and passed through antibacterial filters, and controlled for the presence of the phage.

Stenotrophomonas maltophilia phage strain 11/8c was deposited on Jun. 16, 2010 in the Polish Microbiological Collection, which possesses the international deposit authority status for the purposes of patent proceedings (IDA). The patent deposit was given the accession number PCM F/00056

### EXAMPLE 12

### Phage Stenotrophomonas maltophilia 12/8c

Virulent bacteriophage isolated from concentrated raw sewage from a sewage treatment plant in Opole on S. maltophilia strain 8c.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 8c. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 8c and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 12/8c was deposited on Jun. 16, 2010 in the Polish Microbiological Collection, which possesses the international deposit authority status for the purposes of patent proceedings (IDA). The patent deposit was given the accession number PCM F/00057.

### EXAMPLE 13

### Phage Stenotrophomonas maltophilia 13/27660

Virulent bacteriophage isolated from raw sewage from a sewage treatment plant in Opole on S. maltophilia strain 27660.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 27660. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 27660 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 13/27660 was deposited on Jun. 16, 2010 in the Polish Microbiological Collection, which possesses the international deposit authority status for the purposes of patent proceedings (IDA). The patent deposit was given the accession number PCM F/00058

### EXAMPLE 14

### Stenotrophomonas maltophilia Phage 14/27660

Virulent bacteriophage isolated from concentrated raw sewage from a sewage treatment plant in Opole on S. maltophilia strain 27660.

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 27660. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 27660 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 14/27660 was deposited on Jun. 16, 2010 in the Polish Microbiological Collection, which possesses the international deposit authority status for the purposes of patent proceedings (IDA). The patent deposit was given the accession number PCM F/00059

### EXAMPLE 15

### Stenotrophomonas maltophilia Phage 15/27660

Virulent bacteriophage isolated from concentrated sewage from a sewage treatment plant in Opole on S. maltophilia strain 27660

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 27660. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 27660 and incubated for 18 h at 37° C.The resulting phage lysate was centrifuged for 30 min. at 4.5 KRPM and passed through antibacterial filters, and controlled for the presence of the phage.

Stenotrophomonas maltophilia phage strain 15/27660 was deposited on Jun. 16, 2010 in the Polish Microbiological Collection, which possesses the international deposit authority status for the purposes of patent proceedings (IDA). The patent deposit was given the accession number PCM F/00060

### EXAMPLE 16

### Stenotrophomonas maltophilia Phage 16/27660

Virulent bacteriophage isolated from sewage from a treatment plant in Opole on S. maltophilia strain 27660

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 27660. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 27660 and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 16/27660 was deposited on Jun. 16, 2010 in the Polish Microbiological Collection, which possesses the international deposit authority status for the purposes of patent proceedings (IDA). The patent deposit was given the accession number PCM F/00061

### EXAMPLE 17

### Phage Stenotrophomonas maltophilia 17/8c

Virulent bacteriophage isolated from sewage from the treatment plant in Opole on S. maltophilia strain 8c

Phage isolation was carried out after 1 h of incubation at a temperature of 37° C. from the examined sewage sample in a liquid nutritive medium (at a 1:1 ratio) with the addition of 0.1 ml of a fresh culture of S. maltophilia strain 8c. Next, 0.2 ml of the examined mixture were sampled and loaded with a spreader onto the surface of a plate with a selective agar medium. The plate was incubated for 18 h at a temperature of 37° C. The phage line was derived from individual colonies using an inoculating loop. The colonies were transferred into tubes with a nutritive broth containing 0.05 ml of a fresh culture of S. maltophilia strain 8c and incubated for 18 h at 37° C. The resulting phage lysate was centrifuged for 30 min. at 4.5 KRPM and passed through antibacterial filters, and controlled for the presence of the phage.

The Stenotrophomonas maltophilia phage strain 17/8c was deposited on Jun. 16, 2010 in the Polish Microbiological Collection, which possesses the international deposit authority status for the purposes of patent proceedings (IDA). The patent deposit was given the accession number PCM F/00062

1. A bacteriophage strain specific against bacteria belonging to the genus Stenotrophomonas selected from among strains deposited in the Polish Microbiological Collection under the accession numbers: PCM F/00046, PCM F/00047, PCM F/00048; PCM F/00049; PCM F/00050; PCM F/00051; PCM F/00052; PCM F/00053; PCM F/00054; PCM F/00055; PCM F/00056; PCM F/00057; PCM F/00058; PCM F/00059; PCM F/00060; PCM F/00061 or PCM F/00062.

2. The use of a bacteriophage strain selected from among strains deposited in the Polish Microbiological Collection under the accession numbers: PCM F/00046, PCM F/00047, PCM F/00048; PCM F/00049; PCM F/00050; PCM F/00051; PCM F/00052; PCM F/00053; PCM F/00054; PCM F/00055; PCM F/00056; PCM F/00057; PCM F/00058; PCM F/00059; PCM F/00060; PCM F/00061 or PCM F/00062 in the production of antibacterial preparations for use in combating bacteria belonging to the genus Stenotrophomonas.

3. A use according to claim 2, characterised in that the counteracted bacteria belong to the species Stenotrophomonas maltophilia.
